(19) United States
(12) Patent Application Publication
(10) Pub. No.: US 2008/0009802 A1
(43) Pub. Date: Jan. 10, 2008

(54) Method of treating acne with stratum corneum piercing device

(76) Inventors: Danilo Lambino, Kogarah (AU); Jue-Chen Liu, Belle Mead, NJ (US); Ying Sun, Belle Mead, NJ (US); Justin McDonough, Flemington, NJ (US); Jeffrey M. Wu, Warrington, PA (US); Mariam Maghribi, Fremont, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/113,937

(22) Filed: Apr. 25, 2005

(51) Int. Cl. A61M 5/00

(52) U.S. Cl. 604/173; 604/46; 604/500

(57) ABSTRACT

The invention features a method of treating acne by piercing the stratum corneum of skin in need of such treatment with a stratum corneum-piercing device including a microprotrusion member having a skin-contacting surface and plurality of stratum corneum-piercing microprotrusions thereon.

### BACKGROUND OF INVENTION

Devices have been used for the systemic delivering of active substances through the skin which otherwise would have to be administered intravenously. In particular, transdermal delivery of actives (including patches that deliver nicotine, scopolamine, nitroglycerin, estrogen, and various pain relievers) are quite popular as they allow the user to maintain a steady state of drug delivery. Devices have also been used for single dose delivery or sampling of biological fluids from barrier membranes (e.g., skin). Such devices include those that pierce the skin, thereby disrupting the barrier that the skin provides. In such a puncture-type system, a needle may also be used to deliver systemic drugs into or below the layers of the skin. Examples of these delivery systems are disclosed in U.S. Pat. Nos. 5,879,326, 6,132,755, and 6,743,211.

The present invention provides for devices and/or the use of the devices, for example for the treatment of skin disorders, such as acne.

### SUMMARY OF THE INVENTION

In one aspect, the present invention features a method of treating a skin disorder with a device. In one embodiment, the device includes (i) a microprotrusion member having a skin-contacting surface and plurality of stratum corneum-piercing microprotrusions thereon and (ii) a composition for treatment of the skin disorder, wherein the method includes piercing the stratum corneum of the skin with the microprotrusion member and applying the composition from the device to the skin.

In one aspect, the invention features a method of treating acne by piercing the stratum corneum of skin in need of such treatment with a stratum corneum-piercing device including a microprotrusion member having a skin-contacting surface and plurality of stratum corneum-piercing microprotrusions thereon.

In one aspect, the present invention features a method of removing pus from a pimple by piercing the pimple with a stratum corneum-piercing device, the device including a microprotrusion member having a skin-contacting surface and plurality of stratum corneum-piercing microprotrusions thereon.

In one aspect, the present invention features a device including (i) a microprotrusion member having a skin-contacting surface and plurality of stratum corneum-piercing microprotrusions thereon and (ii) a composition including an anti-acne agent.

In one aspect, the present invention features a stratum corneum-piercing device including a microprotrusion member having a skin-contacting surface and plurality of stratum corneum piercing microprotrusions thereon, the device being adapted to move the microprotrusion member lateral to the surface of the skin surface upon contact with the skin. Examples of lateral movement include, but are not limited to, linear and rotational motion.

Other aspects, features, and advantages of the present invention will be apparent from the detailed description of the invention and from the claims.

### DETAILED DESCRIPTION OF THE DRAWINGS

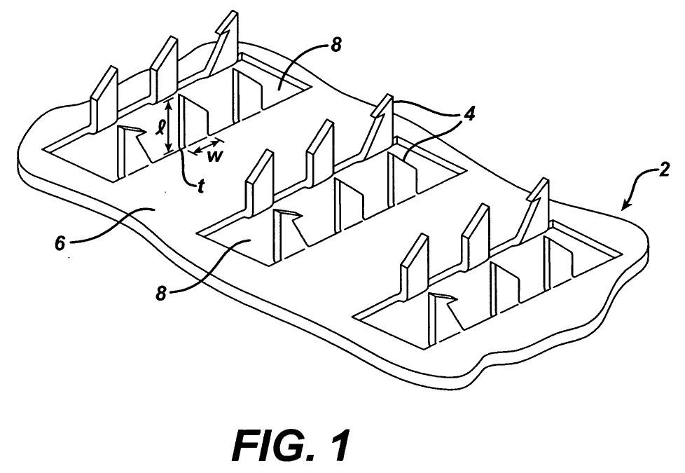

FIG. 1 is an enlarged perspective view of the skin proximal side of a microprotrusion member useful in the present invention;

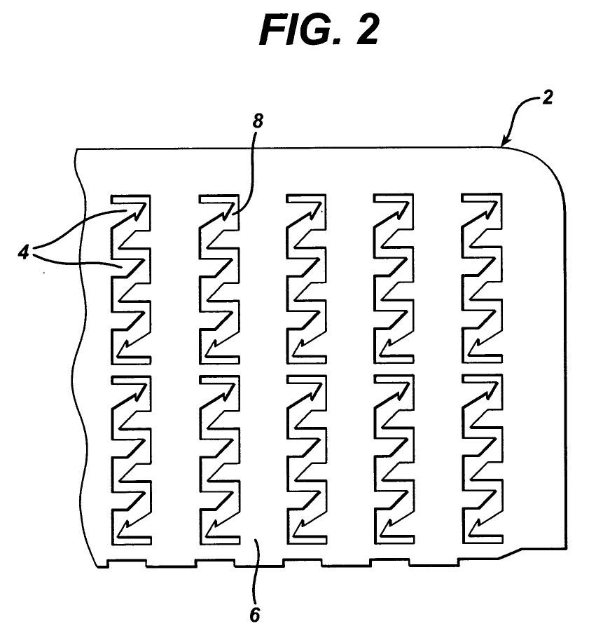

FIG. 2 is a partial top plan view of a microprotrusion member of FIG. 1, before bending/punching the microprotrusions out of the plane of the sheet;

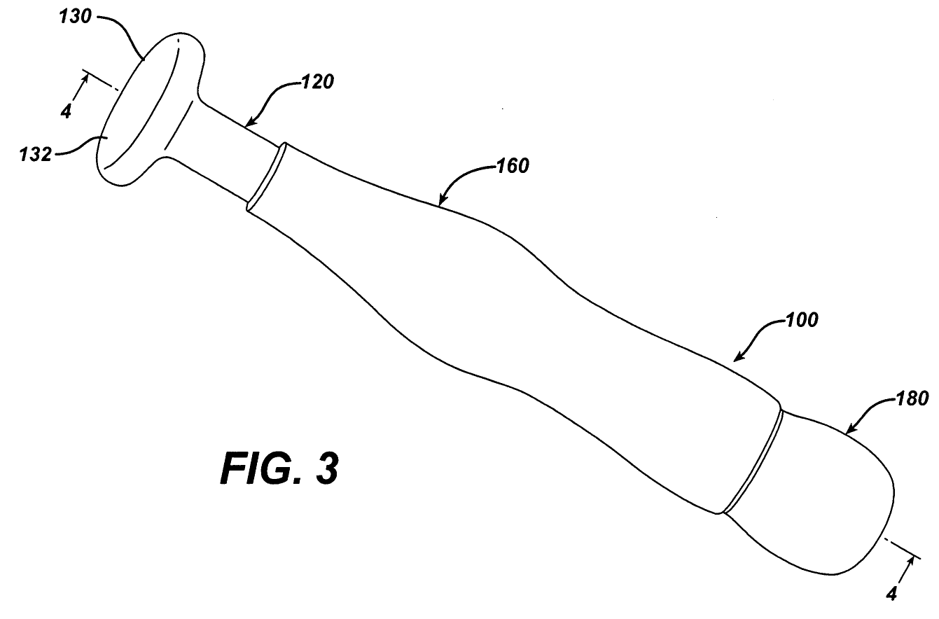

FIG. 3 is a plan view of an implement having a convex skin-contacting surface useful in the present invention;

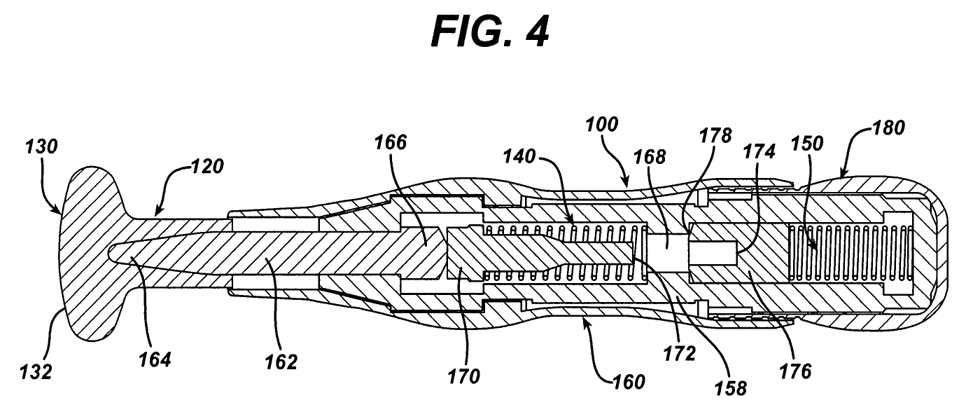

FIG. 4 is a cross sectional view of the implement shown in FIG. 3;

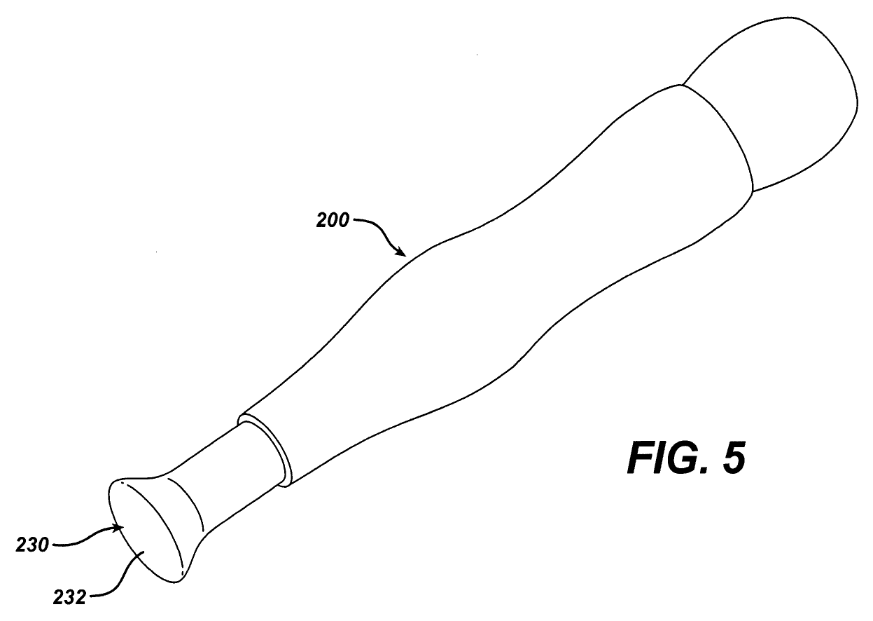

FIG. 5 is a plan view of another embodiment of an implement useful in the present invention;

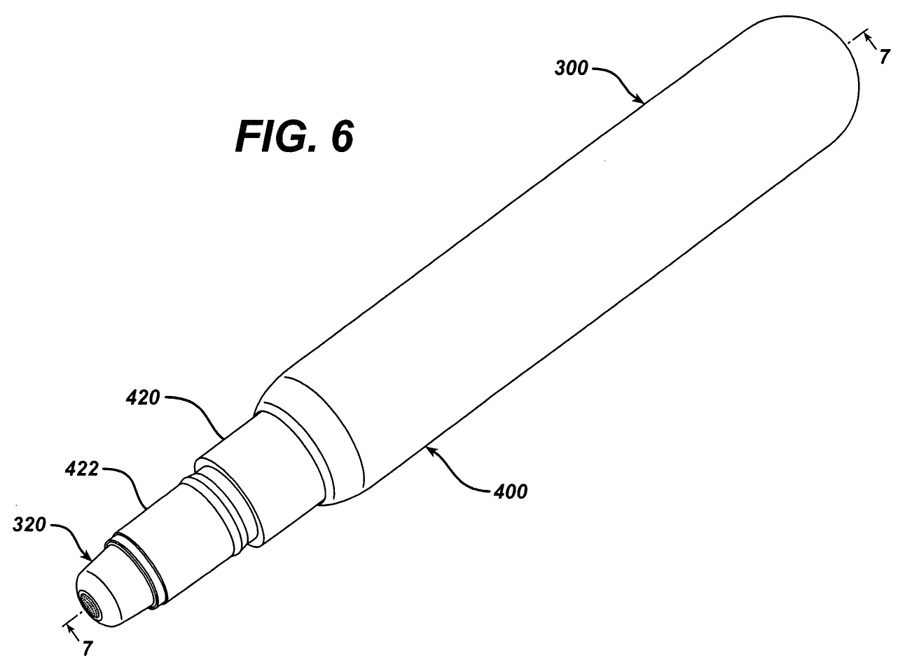

FIG. 6 is a perspective view of another embodiment of an implement useful in the present invention;

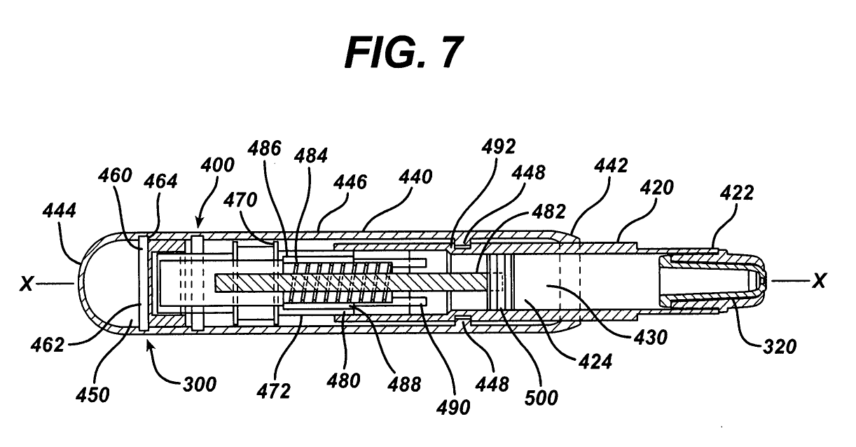

FIG. 7 is a cross-sectional view of the implement shown in FIG. 6;

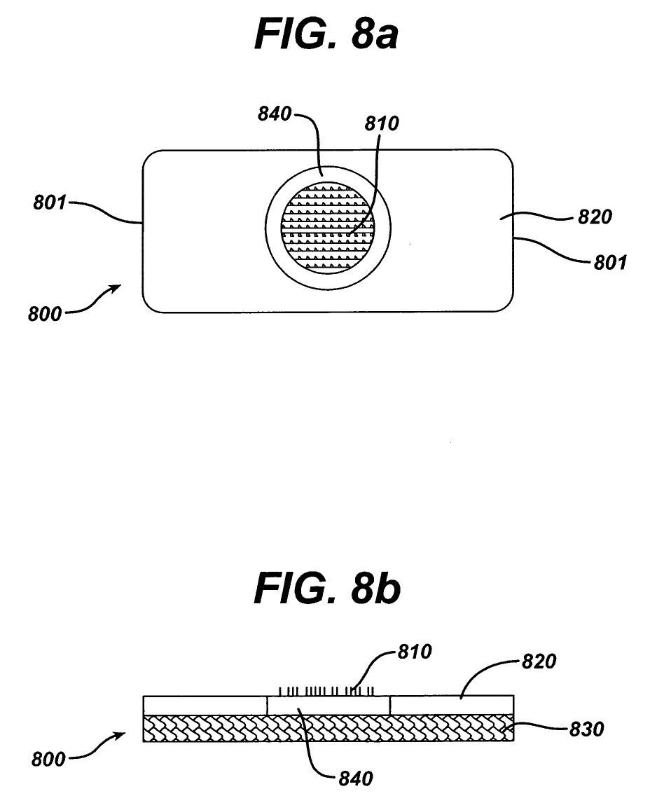

FIG. 8a is a top view of a patch device of the present invention;

FIG. 8b is a cross-section view of a patch device of the present invention;

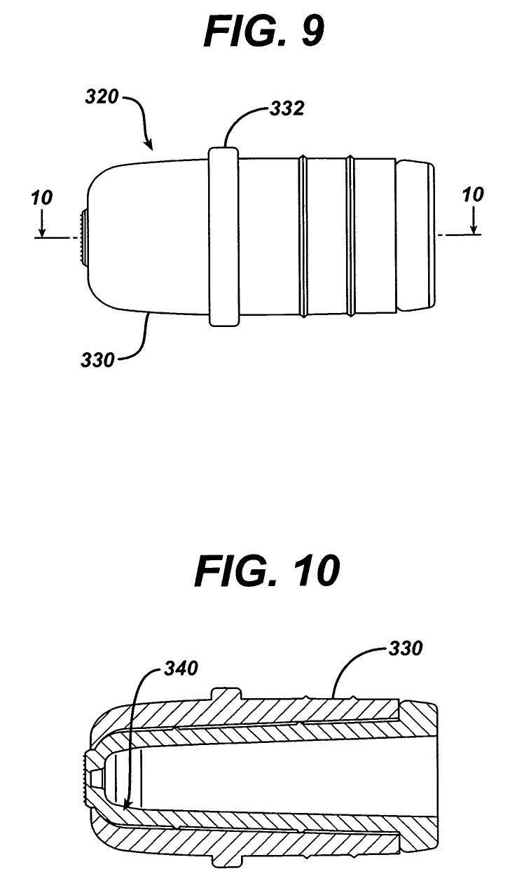

FIG. 9 is a plan view of the microprotrusion member shown in FIG. 7;

FIG. 10 is a cross-sectional view of the microprotrusion member shown in FIG. 9; and

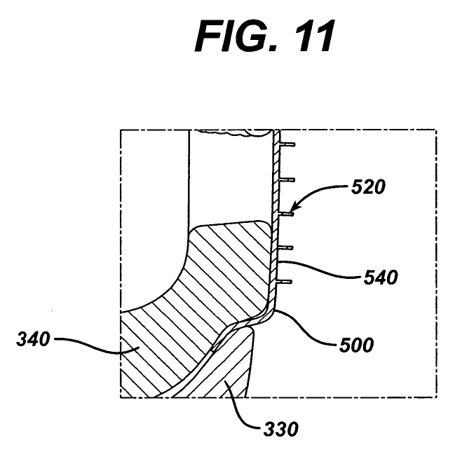

FIG. 11 is a partial view of the microprotrusion member of FIGS. 9-10.

### DETAILED DESCRIPTION OF THE INVENTION

It is believed that one skilled in the art can, based upon the description herein, utilize the present invention to its fullest extent. The following specific embodiments can be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

In one embodiment, the present invention is directed to a device and the use of that device for treating skin disorders, such as acne, scars, or visible skin discolorations. The treatment involves disrupting the stratum corneum of the skin and may or may not further include the application of a composition that permeates into the disrupted skin. A benefit of such a treatment includes localizing the treatment to a certain area of skin in need of such treatment.

Definitions

What is meant by a “product” is a product in finished packaged form. In one embodiment, the package is a container such as a plastic or cardboard box for storing such device and/or kit. In one embodiment, the product contains instructions directing the user to apply the microprotrusion member to the skin (e.g., for the treatment of a skin disorder).

What is meant by “promoting” is promoting, advertising, or marketing. Examples of promoting include, but are not limited to, written, visual, or verbal statements made on the product or in stores, magazines, newspaper, radio, television, internet, and the like. For promoting the treatment of the skin disorder acne, examples of such statements include, but are not limited to, “treats acne,” “safely pops pimples,” “eliminates acne and/or pimples/blemishes”, “visibly reduces the symptoms and/or appearance of pimples”. Similar statements can be made for other skin disorders.

As used herein, “administering to the skin in need of such treatment” means contacting (e.g., by use of the hands or an applicator) the area of skin in need such treatment. These features may be present on the face, such as under or adjacent the eyes, nose, forehead, cheeks, jawls, and neck, as well as other areas of the body such as the arms, chest, back, shoulder, belly (e.g., stretch marks), and legs (e.g., cellulite).

The term “treating” or “treatment” of a skin disorder means the treatment (e.g., complete or partial alleviation or elimination of symptoms and/or cure) and/or prevention or inhibition of the skin disorder.

As used herein, “composition” means a composition suitable for administration to the skin.

As used herein, “cosmetically-acceptable” means that the ingredients or compositions which the term describes are suitable for use in contact with the skin without undue toxicity, incompatibility, instability, irritation, allergic response, and the like. This term is not intended to limit the ingredient/composition to which it describes for use solely as a cosmetic (e.g., the ingredient/composition may be a pharmaceutical agent).

As used herein, “safe and effective amount” means an amount of the active agent, compound, carrier, or of the composition sufficient to induce the desired effect, but low enough to avoid serious side effects. The safe and effective amount of the compounds or composition will vary with the area being treated, the age, health and skin/tissue type of the end user, the duration and nature of the treatment, the specific compound or composition employed, the particular cosmetically-acceptable carrier utilized, and like factors.

Skin Disorder

As used herein, the term “skin disorder” shall mean a disease, disorder, or defect of the skin including, but not limited to, acne (including but not limited to acne vulgaris and acne rosacea), psoriasis, infections, blemishes, hyperpigmentation (including but not limited to post inflammatory hyper-pigmentation (PIH)), hypopigmentation, hair growth disorders such as alopecia and excessive or unwanted hair growth, rough skin, dry skin, lax skin (including but not limited to skin lacking in firmness or elasticity), wrinkles (including but not limited to fine lines and course wrinkles), hypervasculatated skin (including but not limited to dark circles), sebum production disorders (e.g., skin shine), excessive pore appearance, excessive perspiration (including hyperhidrosis), tattoo appearance, rashes (including allergic and diaper), scar appearance, pain, itch, burn, inflammation, warts, corns, calluses, edema, poison ivy/oak, and bites from insects, spiders, snake, and other animals.

Examples of skin infections include, but are not limited to, acne, impetigo, folliculitis, furunculosis, ecthyma, eczema, psoriasis, atopic dermatitis, epidermolysis bullosa, icthyosis, infected traumatic lesions (e.g., ulcers, minor burns, cuts, abrasions, lacerations, wounds, biopsy sites, surgical incisions and insect bites, which have become infected), herpes (e.g., cold sores) or other bacterial or viral infections.

Examples of wrinkled skin include, but are not limited to, fine lines, deep-set wrinkles, laugh lines, crows feet, stretch marks, cellulite, and frown lines.

Examples of discolored skin include but are not limited to hyperpigmented skin, hypopigmented skin, blemished skin, bruised, and hypervaculated skin.

Examples of hyperpigmented skin include, but are not limited to, freckles, age spots (sloar lentigo), sun spots, melasma, sallow color, dyschromia, post-inflammatory pigmentation (PIH), and other discolored skin.

An example of hypopigmented skin includes, but is not limited to, vitiligo.

Examples of blemished skin include, but are not limited to, pustules, comedones, pimples, blackheads or other types of eruptions associated with acne.

Examples of scar skin disorder include, but are not limited to scars from acne, surgery, insect bite, burns, injuries, trauma, and other wounds.

Stratum Corneum-Piercing Device

The stratum corneum-piercing device includes a microprotrusion member having a skin-contacting surface and plurality of stratum corneum piercing microprotrusions thereon. The device may also include one or more reservoirs.

Microprotrusions

The term “microprotrusion” as used herein refers to a stratum corneum piercing element that is adapted to penetrate in the stratum corneum. Microprotrusions typically having a length of from about 20 to about 1000 microns, and preferably from about 50 to about 500 microns, and more preferably from about 100 to about 250 microns. What is meant by length is the length of the microprotrusion adapted to penetrate into the skin (e.g., the length measured from the top of the microprotrusion to the skin-contacting surface or other affixed to the skin contracting surface such as an absorbent reservoir). The average longest diameter (e.g., the width of the microblade or the diameter of a microneedle) measured along the length of the microprotrusions are typically less than half of the length of the microprotrusions, such as less than one quarter of the length of the microprotrusions. In one embodiment, the average diameter of the microprotrusions along its length are from about 5 to about 500 microns, preferably from about 10 to about 250 microns, and more preferably from about 25 to about 100 microns. In one embodiment, the microprotrusions are adapted to penetrate other sections of the epidermis, but are not adapted to penetrate the dermis. However, for certain applications such as treating scars and wrinkles, the microprotrusions may be adapted to penetrate into superficial portions of the dermis.

The microprotrusions may be formed in different shapes, such as needles, hollow needles, blades, pins, punches, and combinations thereof. It is not necessary that the microprotrusions on the device be made of a uniform size (e.g., different lengths or average diameters) or shape. What is meant by the term “blade” or “microblade” is a microprotrusion that has at least one edge. The microblade, optionally, may have a barb.

The term “microprotrusion array” as used herein refers to a plurality of microprotrusions arranged in an array for piercing the stratum corneum. An array of microprotrusions can include a mixture of microprotrusions having, for example, various lengths, outer diameters, inner diameters, cross-sectional shapes, and spacing between the microprotrusions. In one embodiment, microprotrusion array includes hollow needles, for example hollow needles adapted to inject a composition into the skin or remove fluids from the skin.

In one embodiment, the microprotrusion member includes from about 2 to about 5000 microprotrusions, such as from about 10 to about 500 microprotrusions, such as from about 25 to about 200 microprotrusions. In one embodiment, the microprotrusion member has a microprotrusion density of from about 1 microprotrusions/cm² to about 2000 microprotrusions/cm², such as from about 100 microprotrusions/cm² to about 1000 microprotrusions/cm².

Examples of microprotrusion arrays and methods of making same are described in U.S. Pat. Nos. 5,879,326, 3,814,097, 5,279,544, 5,250,023, 3,964,482, and Re. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, and WO 98/29365. Examples of such methods of manufacture include, but are not limited to, chemical vapor deposition, laser machining, and photolithographic processes.

The microprotrusions can be constructed from a variety of materials that have sufficient strength and manufacturability to produce elements capable of piercing the stratum corneum, such as, glasses, silicons, ceramics, metals, metal alloys, semiconductors, inorganic crystals, organic crystals, polymers, polymer composites, and mixtures or composites thereof.

Examples of metals and metal alloys include, but are not limited to, stainless steel, gold, iron, steel, tin, zinc, copper, platinum, aluminum, germanium, zirconium, titanium and titanium alloys containing molybdenum and chromium, metals or non-metals plated with, gold, rhodium, iridium, titanium, platinum, silver, silver halides, and alloys of these or other metals.

In one embodiment, the microprotrusions are made of piezoelectric material that can change the dimension of the microprotrusion corresponding to applied electricity, such as a piezo-ceramic substance. Such manufacture, in one embodiment, would allow motion of the microprotrusions when an electrical current waveform was supplied to piezo-ceramic substance, thereby increasing the disruption of the stratum-corneum. The electricity supplied to the disrupted area may also accelerate healing and other benefits.

In one embodiment, the microprotrusions are made of a shape memory metal, such as Nitinol, that can change the dimension of the microprotrusion corresponding to temperature change. In one embodiment, the microprotrusion member containing Nitinol is heat-treated and fabricated into a first shape, such as shown in FIG. 1. The microprotrusion member is then be distorted into another shape, such as the shape as shown in FIG. 2 (e.g., for easy storage and/or protection of the microprotrusions). During use, an increase in the device temperature (e.g., from the body temperature upon contact) will restore the microprotrusion member back to its first shape. The use of a Nitinol metal alloy can also be used to generate motion of microprotrusions (e.g., into and/or lateral to the skin). Examples of inorganic and organic crystals include diamond, aluminum oxide, soluble or insoluble salt crystals, and quartz.

Examples of glasses include, but are not limited to, devitrified glass such as “Photoceram” available from Corning in Corning, N.Y.

Examples of rigid polymers include, but are not limited to, natural polymers and synthetic polymers, such as polystyrene, polymethylmethacrylate, polycarbonate, polytetrafluoroethylene, polydivinyl fluoride, polypropylene, polyethylene, “Bakelite”, cellulose and cellulose acetate, ethylcellulose, styrene/acrylonitrile copolymers, styrenebutadiene copolymers, acrylonitrile/butadiene/styrene (ABS) copolymers, polyvinyl chloride and acrylic acid polymers including polyacrylates and polymethacrylates, and composites thereof.

In one embodiment, the microprotrusions are made of a biodegradable/bioabsorbable polymer. In such an embodiment, if the microprotrusion, or portions thereof, break off in the skin, they will biodegrade. In a further embodiment, the microprotrusion includes an active agent. Representative biodegradable polymers include, but are not limited to, polymers of hydroxy acids such as lactic acid and/or glycolic acid such as polylactide, polyglycolide, and polylactide-co-glycolide, polyanhydrides, poly(ortho)esters, polyurethanes, poly(butyric acid), poly(valeric acid), poly(lactide-co-caprolactone), and cyclic olefin copolymers. Representative non-biodegradable polymers include polycarbonate, polymethacrylic acid, ethylenevinyl acetate, polytetrafluoroethylene, and polyesters.

In one embodiment, the microprotrusions are formed of a nonporous solid or a porous solid (with or without a sealed coating or exterior portion), and may be hollow. As used herein, the term “porous” means having pores or voids throughout at least a portion of the microprotrusion structure, sufficiently large and sufficiently interconnected to permit passage of fluid and/or solid materials through the microprotrusion. As used herein, the term “hollow” means having one or more bores or channels (e.g., substantially annular bores) through the interior of the microneedle or microprotrusion structure, having a diameter sufficiently large to permit passage of fluid and/or solid materials through the microneedle/microprotrusion. The bores may extend throughout all or a portion of the needle in the direction of the tip to the base, extending parallel to the direction of the needle or branching or exiting at a side of the needle, as appropriate. The base surface that the microprotrusions are attached to, or integral to, may also provide one or more openings.

The microneedle/microprotrusion can have substantially straight or substantially tapered shafts. A hollow microneedle that has a substantially uniform diameter, which needle does not taper to a point, is referred to herein as a “microtube.” In one embodiment, the diameter of the microprotrusion is greatest at the base end of the microprotrusion and tapers to a point at the end distal the base. The microprotrusion can also be fabricated to have a shaft that includes both a substantially straight (e.g., untapered) portion and a substantially tapered portion.

The microprotrusions can be formed with shafts that have a circular cross-section in the perpendicular, or the cross-section can be non-circular. For example, the cross-section of the microprotrusion can be polygonal (e.g. star-shaped, square, triangular, rectangular), oblong, or another shape. In one embodiment, the shaft has one or more bores.

The microprotrusions can be oriented substantially perpendicular or at an angle to the skin-contacting surface. Preferably, the microprotrusions are oriented substantially perpendicular to the skin-contacting surface so that a larger density of microprotrusions per unit area of skin-contacting surface is provided. An array of microprotrusions can include a mixture of microprotrusion orientations, heights, or other parameters.

Generally, the microprotrusions should have the mechanical strength to resist distortion (such as bending) while being inserted into the skin and while being removed. In one embodiment, the microprotrusion is inserted into the skin a single time. In another embodiment, the microprotrusion is inserted into the skin multiple times at the same or at different sites. In one embodiment, the microprotrusion is hollow and should remain intact for delivery of active agents, or for collection of bodily fluids.

An example of a microprotrusion member having a skin-contacting surface and a plurality of microprotrusions is shown in FIGS. 1 and 2. Looking at FIG. 1, microprotrusion member 2 includes a plurality of microprotrusions 4 (i.e., a microprotrusion array) extending from one surface of a skin-contacting surface 6 (FIG. 1 shows microprotrusion member 2 is in an inverted position to show the microprotrusions). The microprotrusions 4 penetrate the stratum corneum of the epidermis when pressure is applied to the device (i.e., the skin of an animal and particularly a human).

The microprotrusions 4 may be formed from a single piece of material (see FIG. 2, which shows the one piece construction prior to the bending of the microprotrusions out of the plane of the sheet) or separately joined to a skin-contacting surface by any manufacturing method (not shown).

In one embodiment, the microprotrusions 4 and the skin-contacting surface 6 are essentially impermeable or are impermeable to the passage of an agent. In one embodiment, the skin-contacting surface 6 is formed with a multiplicity of openings 8 between the microprotrusions 4 for enhancing the movement of an agent or composition there through (e.g., the composition is delivered into the skin from the microprotrusion member through the holes in the stratum corneum which are made by the microprotrusions 4).

In one embodiment where the device is used to treat acne, when the microprotrusion member forms holes in the pimple or affected area, body fluids, such as pus, may be loosened and/or withdrawn into a reservoir of the microprotrusion member through the perforations formed in the stratum corneum and through the openings in the skin-contacting surface. Similarly, the device of the present invention may be used to facilitate the outward flow of wound exudates thus enhancing wound healing.

In one embodiment, the opening 8 corresponds to the portion of the skin-contacting surface 6 occupied by each of the microprotrusions 4 prior to the microprotrusions 4 being transpositioned into the downward depending position. The number of microprotrusions 4 per opening 8 can be any number, preferably however from about 1 to about 30 microprotrusions per opening and more preferable from about 1 to about 4 microprotrusions per opening. Furthermore, the number of openings 8 per microprotrusion member 2 and the number of microprotrusions per microprotrusion member 2 are independent.

In the embodiment shown in FIG. 1, the microprotrusions 4 have an average thickness (“t”) along the length (“l”) of the microprotrusion, which is much smaller than the average width (“w”) along the length of the microprotrusion.

In one embodiment, the skin site is pre-treated with compositions, such as topical anesthetic, antiseptic cleansing, skin softening agents, or heat or cold treatment.

Skin-Contacting Surface

The skin-contacting surface of the microprotrusion member can also be constructed from a variety of materials, including, but not limited to, metals, ceramics, semiconductors, organics, polymers, plastics, and composites thereof. The skin-contacting surface includes the base to which the microprotrusions are attached or integrally formed. A reservoir may also be attached to the skin-contacting surface. In one embodiment, the skin-contacting surface has at least one opening to allow (i) a composition to move from a reservoir, through the opening, and onto the skin and/or (ii) bodily fluid to move from the skin, through the opening, and into a reservoir. In one embodiment, the skin-contacting surface forms a stop and help control how deep the microprotrusions can penetrate the skin.

In one embodiment of the device, the skin-contacting surface is formed from a thin, rigid material that is sufficiently stiff so as to force the attached microprotrusions through the skin in such areas where the skin resists deformation by the microprotrusions, such as those materials used to form the microprotrusions. Examples include but are not limited to, glasses, silicons, ceramics, metals, metal alloys, semiconductors, inorganic crystals, organic crystals, polymers, polymer composites, and mixtures or composites thereof.

In another embodiment, the skin-contacting surface is formed from flexible materials to allow the device to fit the contours of the skin and to adapt to deformations that may occur when the microprotrusions are applied. A flexible surface further facilitates more consistent penetration during use, since penetration can be limited by deviations in the attachment surface. For example, the surface of human skin is not flat due to dermatoglyphics, e.g., wrinkles, scars, pimples, and hair, and is highly deformable. The flexible skin-contacting surface can be deformed mechanically (for example, using an actuator or other pressure) in order to pierce the skin.

The size of the skin-contacting surface will depend on the area of the skin disorder being treated. In one embodiment, the area of the skin-contacting surface is from about 0.05 cm² to about 500 cm², such as from about 0.1 cm² to about 100 cm². In one embodiment, the total surface area of the one or more openings from about 1 to about 95 percent of the total surface area of the skin-contacting surface (e.g., including the surface area of the opening(s)), such as from about 50 to about 80 percent.

Reservoir

In one embodiment, the device disclosed herein also includes one or more reservoirs for containing one or more compositions and/or collecting body fluids, such as pus or wound extrudate, from the skin.

In one embodiment, the reservoir is in communication with the microprotrusion member. In one embodiment, the reservoir is attached by an adhesive (such as cyanoacrylate glue) to the side of the skin-contacting surface opposite the side including the microprotrusions. A seal lining may also be included to secure the holding of the fluid collected.

The reservoir may be in the form of a chamber enclosed with rigid or flexible walls or in the form of a absorbent substrate such as a nonwoven fabric, a hydrogel, or hydrocolloid pad (e.g., in a bandage-like device with backing layer). The rigid polymer materials that may be used to manufacture the rigid reservoir include but are not limited to natural polymers and synthetic polymers, such as polystyrene, acrylonitrile/butadiene/styrene (ABS) copolymers polymethylmethacrylate, polytetrafluoroethylene, polycarbide, nylon, and polycarbonate. The flexible polymers that may be used to manufacture the flexible polymer reservoir enclosure include but are not limited to as polyethylene, polypropylene, polyurethane, thermoplastic elastomers, silicones, latexes, rubbers, and polyvinyl chloride. Absorbent materials include, but are not limited to, woven and nonwoven materials, hydrogels, and hydrocolloids.

A composition containing benefit agents may be stored in the reservoir prior to administering to the skin. The reservoir may be a pouch, a small bag, a unit-dose container with any shape and size. It may be squeezable to dispense the composition to the skin before, during or after the microprotrusion application. The reservoir may also be connected to a vacuum mechanism, or be able to create a vacuum environment, in order to extract body waste to extract pus from a pimple. See, e.g., U.S. Pat. No. 6,562,014.

In one embodiment, microprotrusion arrays are attached to an extraction device, as described in U.S. Pat. No. 6,562,014 and may be applied to treat pimples or extract the pus from pimples filled with pus (pustule). The plunger of the extractor device is pulled out first and then, the device is placed on the treated skin site. Using the thumb, the plunger is pushed in all the way until the microprotrusions pierce the stratum corneum and a suction action is activated to remove the pus. In one embodiment, a vacuum in the range of from about 0.1 to about 0.8 atm is applied to create plural microchannels. A seal film or liner made from, for example polyurethane, may be added to the extractor opening end to maintain the vacuum. Optionally, a disposable absorbent material made from e.g. cellulose, or nonwoven material, is added behind the microprotrusion disk to collect pus waste from the pimple. Optionally, a topical composition may be applied to the treated site at this point.

In one embodiment, the reservoir may contain an absorbent material such as sodium carboxymethyl cellulose adhesive, a hydrogel or a nonwoven fabric.

Patch

In one embodiment, the microprotrusion membrane in FIG. 1 may be fabricated into an adhesive patch device that resembles a bandage or transdermal patch. In one embodiment, the adhesive patch device 800 (FIGS. 8a and 8b) has a multi-layered device structure: the top layer is the microprotrusion member 810, the second layer is the absorbent layer 820, and the third layer is the backing layer 830. FIG. 8b shows a cross sectional view of the device of FIG. 8a taken along lines 801.

The absorbent layer 820 may be replaced with a non-absorbent layer, which can be made of rigid or flexible materials. In one embodiment, absorbent layer 820 contains a reservoir 5 that contains a composition to be dispensed through the microprotrusion member 810. Reservoir 850 may be made from an individual or multiple chambers.

In one embodiment, there is an adhesive coating at the periphery edge of backing layer 830 in order to affix the patch to the skin of a user (e.g., similar to an island-type bandage design). Alternatively, if the absorbent layer 820 is adhesive hydrogel or hydrocolloid layer, the patch may not require such additional adhesive for skin attachment. In one embodiment, the device 800 includes a release liner layer to cover the device 800 prior to use (not shown). In one embodiment, the absorbent layer 820 in the patch device is used to extract bodily fluids (such as pus from a pimple) after the microprotrusion member of the device pierces the stratum corneum. In one embodiment, a composition in the absorbent layer (or coated on the microprotrusion members) is delivered into the diseased skin after the microprotrusion member pierces the stratum corneum.

The patch device in a sealed in a package during storage. The sealed package may assist the device in remaining sterile and stable by blocking microbiologic pathogens, moisture, oxygen, UV rays, and/or other harmful elements.

In one embodiment, the patch is left on the skin for an extended period of time to deliver the active agent and/or composition into skin or to extract bodily fluids from the treatment site. In one embodiment, the patch is left on the skin for an extended period of time, such as for 5 minutes, 15 minutes, 30 minutes, one hour, 4 hours, or up to 24 hours.

Adhesive

In one embodiment, the stratum-corneum penetrating device contains an adhesive (e.g., on or outside the skin-contacting surface of the microprotrusion member to affix the device to the skin). The adhesive may be coated over the entire skin-contacting surface of the device, or preferably, only over the periphery or selected areas of the skin-contacting surface. Examples of hydrophobic adhesives include, but are not limited to, silicones, polyisobutylenes and derivatives thereof, acrylics, natural rubbers, and combinations thereof. Examples of silicone adhesives include, but are not limited to, Dow Corning 355 available from Dow Corning of Midland, Mich.; Dow Corning X7-2920; Dow Corning X7-2960; and GE 6574 available from General Electric Company of Waterford, N.Y. Examples of acrylic adhesives include, but are not limited to, vinyl (D acetate-acrylate) multipolymers such as Gelva 7371, available from Monsanto Company of St. Louis, Mo.; Gelvao 7881; Gelva 2943; and 1-780 medical grade adhesive available from Avery Dennison of Painesville, Ohio. Examples of hydrophilic adhesives include, but are not limited to, gum papaya and other natural gums, MC, HEMA, HPMC, EHEC, HEC, HPC, CMC, PVA (polyvinyl alcohol), PVP (polyvinyl pyrrolidone), PEO (polyethylene oxide), HEMA, HEEMA, HDEEMA, MEMA, MEEMA, MDEEMA, EGDMA, NVP MA, VAC, polycrylamide, gelatins, gum arabic, gum karaya, gum tragacanth, guar gum, gum benzoin, and alginic acid and their salts, polyethylene glycol (PEG), and polypropylene glycol (PPG).

In one embodiment, the concentration of the adhesive in the adhesive coating layer may range from about 0.1% to about 95%, by weight, such as from about 1% to about 20%, by weight, of the carrier.

Devices

In one embodiment, the microprotrusion member is digitally pushed into the skin by the user (e.g., the fingers of the user exert enough pressure for the microprotrusion member to pierce the stratum corneum).

Finger Cot/Glove

In one embodiment, the stratum-corneum piercing device of the present invention may be constructed as a part of a finger cot or a glove with the microprotrusions facing outwards. By wearing such a finger cot-like or glove-like device, the user can treat the skin with precision and ease, especially at certain anatomic sites that require precision in application (e.g., around the eye) or are difficult to reach (e.g., the back). The microprotrusion member may be located on certain areas of the finger cot-like or glove-like device that would touch the skin (e.g., on the tip area), or may cover the entire surface of the finger cot-like or glove-like device. The device may also be used to administer the composition.

Roller

In one embodiment, the stratum-corneum piercing device may be constructed in the shape of a roller with the microprotrusions facing outwards. The roller-like microprotrusion member may be rolled over the skin to be treated, thus piercing the stratum corneum and delivering the active agents into the skin. The skin treatment composition may be applied to the skin prior to, during, or after the treatment with the roller-like stratum-corneum piercing device, which may or may not have one or more reservoirs containing the composition and/or collection of bodily fluids. Alternative, the stratum-corneum piercing device may be constructed with a curved surface to resemble a portion of a roller (e.g., with a half-cylinder or quarter-cylinder shape) with the microprotrusions facing outwards on the curved surface. During an application, one end of the partial cylinder shaped microprotrusion member is pressed onto the skin first, followed by a pressing and “rolling” motion over the skin area to be treated until reaching the other end of microprotrusion member, thus piercing the skin that has been rolled over with the device. The main advantage of such a partial cylinder shaped device over the roller-like stratum-corneum piercing device is better control of the applied pressure and movement.

Impact Implement

In another embodiment, the user may engage an implement to push the microprotrusion member into the skin. In one embodiment, the stratum-corneum piercing device includes an implement handle device that may include springs, pistons, pump(s), sensor(s), and/or microprocessor(s) to control the interaction of the microprotrusion member with the skin. The implement handle device may include a reservoir, vacuum or positive pressure source (to collect or expel contents to or from the reservoir), springs or other potential energy storage elements, and/or a collar for securing the microprotrusion member.

Turning to FIGS. 3-7, various embodiments of implement handle devices are shown. FIGS. 3 and 4 show one embodiment of a device 100 having a piston assembly 120 including microprotrusion member contacting portion 130, a main housing portion 160, and an end housing portion 180. FIG. 5 shows an alternate embodiment of implement handle device 200. In these embodiments, the implement handle device incorporates two stages to accomplish application of the microprotrusion member. The first stage has dual actions, particularly via its normal force to the skin surface, both to tension the skin and to initiate seating the microprotrusions into the tensioned skin. The second stage provides an impact force, which will seat the microprotrusion member to the proper depth into the skin. The microprotrusion member contacting portion 130 of the implement handle device 100 provides a uniform distribution of the force so that the microprotrusion member penetrates uniformly, that is, the blades penetrate to substantially the same depth across the contacted skin area.

FIG. 4 shows a cross-sectional view of FIG. 3 taken along lines 4-4. The front portion 164 of the piston assembly 120 extends into microprotrusion member contacting portion 130. The rear portion 166 of the piston assembly contacts impact plunger 170. In the embodiment shown in FIG. 4, internal housing is denoted as 158 and the main housing is denoted as 160. It, however, is not necessary that the two housing be separate; in another embodiment the two may be combined to be a single, integral housing component.

To use the handle implement with the microprotrusion member, the microprotrusion member is first placed on the skin to be treated. The microprotrusion member contacting portion 130 of the device is placed over the microprotrusion member and pressure is exerted by the user to set the microprotrusions into the upper stratum corneum. The pressure on the plunger results in translation of the piston assembly 120 and impact plunger 170 generating tension as it pushes against tensioning spring 140. Tensioning spring 140 may be a straight spring or a conical spring. The position of impact plunger 170 is eccentric or skewed, such that as pressure is applied to the piston assembly, distal end 172 of impact plunger 170 engages edge 178 of impact hammer 176. Once impact hammer 176 is engaged, piston assembly 120, impact plunger 170, and impact hammer 176 continue to translate together until impact plunger 170 becomes aligned through plunger guide 168 as the impact plunger “pops” into the impact hammer hole 174. As this occurs, the plunger and hammer become aligned and impact hammer 176 is forced via impact tension adjustment spring 150 in the opposite direction over the end of impact plunger 170 substantially the length of the impact hammer hole 174 and thereby creating an impact force. The impact force results in an audible noise similar to a click and also an impact perception from microprotrusion member contacting portion 130. When the implement handle device 100 is removed from skin, it will automatically reset itself and be ready for the next operation.

In this embodiment, there are two springs contained within housing of the implement handle device, allowing the skin to be tensioned each and every time the implement is used. In one embodiment, the microprotrusion member is placed on the skin to be treated and set into the skin by using implement 100. Alternately, the microprotrusion member may be affixed to the surface 132 of the microprotrusion member contacting portion 130 of implement 100. The user would then bring the microprotrusion member in contact with the skin surface and push the implement toward the skin surface, thereby setting the microprotrusion member into the skin.

The microprotrusion member contacting portion 130 of FIG. 3 has surface 132 that may be substantially convex (shown FIG. 4), concave, or flat. In the implement device handle 200 shown in FIG. 5, surface 232 of the skin-contacting portion 230 is substantially flat.

The amount of force needed to set the microprotrusions into the skin can vary by skin site or the structure of the microprotrusions. For example, the skin of the elbow is thicker than the skin under the eye and may require a greater force to penetrate into the stratum corneum. In one embodiment, the implement provides at least about one pound of force to force the microprotrusion member into the stratum corneum, such as from about 1 to about 10 pounds of force.

Rotational Device

In another embodiment shown in FIGS. 6 and 7, the microprotrusion member 320 is incorporated as a part of the implement handle device 400 to form a stratum-corneum piercing device 300. Microprotrusion member 320 is shown in greater detail in FIGS. 9 to 10. The microprotrusion member can also be set at an angle to the longitudinal axis (not shown).

Looking at FIG. 6, stratum-corneum piercing device 300 is formed by microprotrusion member 320 and implement handle device 400, which has a rotating/sliding barrel 420. The microprotrusion member 320 is detachably secured into the first end 422 of rotating/sliding barrel 420. Microprotrusion member 320 may be adapted to be removed and replaced by the user whenever desired. A cover, such as a removable cap (not shown) may also be used to cover microprotrusion member 320.

FIG. 7 shows the cross-sectional view of microprotrusion member 300 along line 7-7. Ring 332 of microprotrusion member 320 is in juxtaposition to first end 422 of rotating/sliding barrel 420 and forms an insertion stop. Housing 440 forms the major portion of implement handle device 400. Rotating/sliding barrel 420 is positioned substantially within housing 440 at first end 442. The outer diameter of rotating/sliding barrel 420 is such that barrel 420 is able to slide back and forth without excessive drag but is such that the fit is fairly tight and barrel 420 does not shift in its movement or direction about longitudinal axis X-X (e.g., barrel 420 remains substantially coincidental to housing 440). Housing 440 has first end 442 and second end 444. In the embodiment shown in FIGS. 6 and 7, second end 444 is a rounded end but may be any configuration including flat, convex, or open. On the interior surface 446 of housing 440, there may be stops or notches to hold springs, gears, and plungers. In the embodiment shown in FIGS. 6 and 7, stop 450 is located on the interior surface 446 toward second end 444. The first end 462 of stationary end gear 460 engages stop 450. End gears 460 can also be made integral to housing 440. Second end 464 of stationary end gear 460 engages end cap 470. The interface between the stationary end gear 460 and end cap 470 may include intermeshing teeth, which provides ratcheting during rotation of end cap 470. Within end cap 470, rotating and sliding gear 480 is inserted. Gear 480 has compression spring 484 about shaft 482. Shaft 482 is aligned with and fits into collar 472, which is integral to end cap 470. This arrangement forms a stop for first end 486 of compression spring 484. Second end 488 of compression spring 484 fits into rotating sliding gear 480 which then fits into front stationary gear 490 to form stop 492. Shaft 482 extends through front stationary gear 490 to contact plunger 500. In the embodiment shown in FIG. 7, shaft 482 is threaded, with stationary gear 490 movable about the threads in shaft 482 and mating threads in gear 490. This allows plunger 500 to move toward rotating/sliding barrel first end 422 as the microprotrusion member is applied to the skin.

As previously mentioned, rotating/sliding barrel 420 fits within first end 442 of housing 440. Rotating/sliding barrel 420 has at least one, preferably two or more, rotational grooves shown as 424. Barrel 420 is also preferably substantially clear such that the amount of composition within the barrel can be visualized by the user. Engaging rotational groove 424 is key 448 on the inner surface of housing 440. In the embodiment shown in FIG. 7, there are two keys 448. In the embodiment shown in FIG. 7, groove 424 threads in a helical direction. When groove 424 engages key 448, the movement of the rotating/sliding barrel is also in a helical manner, that is, the rotating/sliding barrel extends away from second end 444 while slightly turning. Within rotating/sliding barrel 420 is reservoir 430. Plunger 500 may engage any composition contained within reservoir 430, thereby expelling the contents through microprotrusion member 320. In the embodiment shown in FIGS. 6 and 7, the plunger incrementally advances toward first end 422 with each successive application. In one embodiment, the motion results in the microprotrusion member 320 being rotated. In another embodiment, the motion results in the microprotrusion member(s) being translated or translated and rotated. In one embodiment, an audible sound is also produced. In another embodiment, a light indicator or other indicator is utilized. In another embodiment, the helical action described in this invention may be precisely and automatically, controlled by a electrical motor (not shown). A circuitry and/or power source for such motor can also be housed inside, e.g. inside implement device 400.

In one embodiment, the user first contacts microprotrusion member 320 with the skin by holding microprotrusion member 320 in a generally perpendicular manner to the skin surface. The user then gently pushes the microprotrusion member into the skin. By applying a force greater than that required by compression spring 486 to compress, the microprotrusion member penetrates the stratum corneum. As the pressure is exerted by the user, the microprotrusion member and barrel 420 translates and rotates through and about the longitudinal axis X-X of the implement handle device 400, moving the microprotrusion member in a circular manner relative to the surface of the skin; that is, the microprotrusion member rotates while contacting and/or entering the skin.

This type of penetration provides a larger pierced area than an area that has just had the microprotrusion member applied to in a non-rotated manner. Use of an implement such as described to set a microprotrusion member into the skin may provide repeatable function and penetration of the microprotrusions into the stratum corneum. The microprotrusion member then resets with an audible click for the next use. The thread pitch and cross-sectional area of the plunger control the amount of composition applied to the skin.

The microprotrusion member of the device is adapted to rotate about 70 degrees lateral to the surface of the skin. In one embodiment, the amount of rotation of the device may be designed to be at least about 5 degrees, such as from about 20 to about 360 degrees, such as from about 45 to about 135 degrees.

An advantage to the embodiment shown in FIGS. 6 and 7 is that a composition that is delivered from reservoir 430 is positively displaced by the plunger at the same time as the stratum corneum is pierced from the same device.

In one embodiment, the stationary end gear 460 and the bottom of the end cap 470 intermeshes and allows for one way rotation of the end relative to the outer housing.

The microprotrusion member 320, thus, allow controlled piercing of the stratum corneum, pressure, torque, rotation, and dispensing of a specific amount of composition to the skin. For the device shown in FIGS. 6 and 7, the microprotrusion member 320 is applied to the skin, the rotating/sliding barrel assembly (including rotating barrel 420, microprotrusion member 320, front stationary gear 490, plunger 500, threaded shaft 482, and rotating/sliding gear 480), translates along the longitudinal axis of the housing assembly (including housing 440, end cap 470, and stationary end gear 460). During this translation, the rotating/sliding barrel assembly also rotates about the longitudinal axis X-X of the housing assembly, with the exception of the threaded shaft 482 and the rotating sliding gear 480. That is to say the threaded shaft 482 and the rotating/sliding gear 480 remain rotationally fixed to housing assembly during this first stage of motion.

The end cap 470 is held fixed during this stage of motion due to a one-way rotation, mating ratchet configuration with the first end 462 of stationary gear 460. The end cap 470 restricts the rotation of both the rotating/sliding gear and the threaded shaft 482. That is to say the threaded shaft 482 and the rotating/sliding gear 480 remains rotational fixed to the end cap in this device.

As a result of the described motion above, front stationary gear 490 rotates relative to both the mating threaded shaft 482 and the sliding/rotating gear 480. This relative rotation between the threaded shaft 482 and the front stationary gear 490, results in translation of the shaft 482 and plunger 500 relative to the rotating sliding barrel 420, pushing out a measured dose of product from the reservoir 430.

The relative rotation between the sliding/rotating gear 480 and the front stationary gear 490 is restricted to one-way rotation, due to a mating ratchet configuration between the sliding/rotating gear 480 and the front stationary gear 490. As the end of the translation and rotation stroke is approached, the front stationary gear 490 ratchets pop over the corresponding sliding/rotating gear ratchet, creating a signal to notify the user that the limit of rotation, translation, and pressure for this application has been reached.

During the described motion above, the spring 484 is compressed, providing a measurable and controllable force measured at the surface contact area. This compressed spring force also maintains engagement of the mating component mating areas for constant engagement of ratchets mating surfaces during translation and rotation.

The relative helical motion between the rotating/sliding barrel assembly and the housing assembly is created through incorporation of a helical groove(s) 424 located in the barrel 420 and the mating key(s) 448 in the housing 440. This motion, however, could also be created through many methods know in the art such as rack and pinion, ball screw, mating screws, etc. Another embodiment includes the key being located on the barrel and the grooves being located in the housing.

The second motion and method of action describe here in occurs with the removal of the device from the contact surface area. At this point in time the rotating/sliding barrel assembly has substantially reached its designed motion limit within the housing, and the spring 484 is substantially compressed.

As the user begins to remove the device from the area of contact, the user motion is opposite to the contact area. As this occurs, the rotating/sliding barrel assembly translates along the longitudinal axis X-X of the housing assembly, remaining in contact with the contact surface. During this translation, the rotating/sliding barrel assembly also rotates about the longitudinal axis X-X of the housing assembly opposite the application rotation, this time including the threaded shaft 482 and the rotating sliding gear 480. That is to say that the threaded shaft 482 remains substantially fixed in position to the mating threads of the front stationary gear 490, allowing only one-way translation of the shaft and piston relative to the barrel reservoir 430, minimizing potential contamination of the device from external contaminants. This rotation also provides a controlled spreading of dispensed product over and/or into the contacted area.

The end cap 470 rotates substantially with the rotating/sliding barrel assembly, during this motion. The mating ratchet configuration with the stationary gear 460 allows one-way rotation in this direction. When the translation limit is reached, the ratchet(s) of the end cap 470 jumps over the corresponding ratchet(s) of the end stationary gear 460, providing a click to notify the user that the device is reset and ready for the next application. The compression spring 484 is either fully extended or at it minimal compressed state at this point.

The implement may be made from a variety of suitable materials. In one embodiment, the plunger 500 is made from a softer material than the shaft 482. For example, in one embodiment, plunger 500 is made from a low-density polyethylene while the shaft is made from an acetal copolymer.

FIGS. 9-10 show in detail one embodiment of microprotrusion member 320 (FIG. 10 is a cross-section view of microprotrusion member 320 along line 10-10). Microprotrusion member 320 has outer housing 330, which includes ring 332. As seen in more detail in FIG. 11, inner housing 340 fits within outer housing 330 and secures microprotrusion member 500. Microprotrusion member 500 contains microprotrusions 520 and skin-contacting surface 540.

In another embodiment (not shown), the implement is a stick-like structure that does not have any gears or rotational ability. The microprotrusion member may be again placed on the skin with the implement used to set the microprotrusions into the skin. Alternately, the microprotrusion member may be attached to an end of the implement. The user would then grip the implement and push the microprotrusion member into the skin. The implement may have any shape. In one embodiment, the outer surface of the implement can be seen in FIG. 6 but have no movable internal parts.

The device can also be designed to create a negative pressure/vacuum for removal of fluid upon contact to the surface or externally triggered by the user. In one embodiment, this can be done in either a single or two step process. In an example of a single step process, the device is applied to the skin as previously described. The motion causes the plunger 500 to recede into the reservoir away from the tip creating a vacuum or negative pressure at the tip. The amount of vacuum created is a function of the amount of air displaced. The reservoir in this case is a vacuum reservoir, not to be used for composition delivery.

In an example of a two-step process, the user would be required to reset the device prior to engaging the contact surface. An example of this would allow the user to push in or pull back a lever to store the required potential through a spring or other potential energy storage device. Then the device would be applied to the contact area, the potential energy would be released creating the motion necessary to produce the vacuum. This embodiment would allow isolation of the force required for application of the micro protrusion to create the punctures and the force required to create the vacuum.

In both the single and two-step process listed above, the mechanical energy/action provided by the user could be replaced by using stored electrical energy to drive a motor (linear or rotary) to create the desired motion of the piston and, therefore, the vacuum. A vacuum pump could also be used.

The device could further be designed to incorporate both a composition reservoir(s) and a vacuum reservoir for both removal of liquid and application of composition from the same device. Concentric or “side by side” reservoirs could be utilized with separate plungers to both create the vacuum and dispense the composition within the same device.

Composition

The composition may be solid, semisolid, liquid or any combination thereof. In particular, the solid compositions include but are not limited to bars, sticks, powders, masks, and patches. Examples of semisolid compositions include but are not limited to creams, lotions, gels, ointments, hydrogels, hydrocolloids, foams, mousses, emulsions, and micro-emulsions. Examples of liquid compositions include but are not limited to cleansers, toners, serums, liquid sprays, and aerosols. Included are those compositions used to treat the aforementioned skin disorders. The composition may contain an active agent (e.g., contains a cosmetically-acceptable, safe and effective amount of such active agent).

In one embodiment, a composition is applied to the skin prior to piercing of the stratum corneum by the microprotrusion member. The composition is then “pushed” into the openings in the skin as the microprotrusion member pierces the skin. In another embodiment, the skin treating composition is present on the microprotrusion member. The composition may be coated on the microprotrusions and/or the skin-contacting surface. One example of a coating is described in European Patent No. 914,178. In this embodiment, the composition may be pushed into the skin as the openings are formed or may “fill in” the openings after they are formed. It has been found that the openings close up within a relatively short time period after forming. Thus, in one embodiment, the coatings are optimized such that as the impact force of the microprotrusion member both pierces the stratum corneum and delivers the composition or active agent to the skin. In one embodiment, the composition is applied to at least a portion of the surface microprotrusion member proximate to the time of application to the skin.

In still another embodiment, the skin treating composition is contained in the reservoir of the microprotrusion member or handle implement. In this embodiment, the composition may be pushed into the skin during penetration or placed on the skin after penetration.

In one embodiment, the composition contains one or more active agents. What is meant by an “active agent” is a compound (e.g., a synthetic compound or a compound isolated from a natural source) that has a cosmetic or therapeutic effect on the body (e.g., a material capable of exerting a biological effect on the skin) such as therapeutic drugs, including, but not limited to, organic and macroionmolecular compounds. Examples of such therapeutic drugs include peptides, polypeptides, proteins, and nucleic acid materials containing DNA; and nutrients. Examples of polypeptide and protein active agents include growth hormone releasing factor (GRF), nerve growth factor, melanocyte inhibitor-I, vaccines, botox (Botulinum neurotoxins), cyclosporin and its derivatives (e.g., biologically active fragments or analogs). Other active agents include anesthetics; analgesics (e.g., lidocaine, lidocaine plus epinephrine, prilocaine, tetracaine, fentanyl, and salts thereof such fentanyl citrate); anti-inflammatory agents; antibiotics, antifungals, antiviral and other antimicrobial agents; antioxidants; immunosuppressive agents and immunostimulants.

In one embodiment, the composition contains an anti-acne agent. What is meant by an anti-acne agent is an compound that has been approved by the U.S. Food and Drug Administration for the topical treatment of acne and/or rosacea. Examples of anti-acne agents include, but are not limited to, salicylic acid, azaleic acid, benzoyl peroxide, sulphur, retinoic acid, tazarotene, candida bombicola/glucose/methyl rapeseedate ferment, peat water, resorcinol, silt, peat, permethin, clindamycin, adapalene, erythromycin, sodium sulfacetamide, and combinations thereof. In one embodiment, the amount of anti-acne agent in the composition is from about 0.01% to about 10%, for example from about 0.1% to about 5%, or from about 0.5% to about 2% by weight, based on the total weight of the composition.

In one embodiment, the device of the present invention contains an anti-aging agent. Examples of suitable anti-aging agents include, but are not limited to: inorganic and organic sunscreens such as titanium dioxide, zinc oxide, and octyl-methoxy cinnamates; retinoids; botox (Botulinum neurotoxins); dimethylaminoathanol (DMAE); copper containing peptides; vitamins such as vitamin E, vitamin A, vitamin C, and vitamin B and vitamin salts or derivatives such as ascorbic acid di-glucoside and vitamin E acetate or palmitate; alpha hydroxy acids and their precursors such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, isopropyl pyruvate, methyl pyruvate, mucic acid, pyruvic acid, saccharic acid, saccaric acid 1,4-lactone, tartaric acid, and tartronic acid; beta hydroxy acids such as beta-hydroxybutyric acid, beta-phenyl-lactic acid, and beta-phenylpyruvic acid; zinc and zinc containing compounds such as zinc oxides; and botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, and safflower; and salts and prodrugs thereof.

In one embodiment, the composition contains a depigmentation agent. Examples of suitable depigmentation agents include, but are not limited to: hydroquinone; lignin peroxidase; mushroom enzymes; hydrogen peroxide; diodic acid; discetyl bolidine; undecylenoyl phenylalanine; glutathione reductase; soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; and salts and prodrugs thereof.

In one embodiment, the composition contains a plant extract. Examples of plant extracts include, but are not limited to, feverfew, soy, glycine soja, oatmeal, what, aloe vera, cranberry, hazel witch, alnus, arnica, artemisia capillaris, asiasarum root, birch, calendula, chamomile, cnidium, comfrey, fennel, galla rhois, hawthorn, houttuynia, hypericum, jujube, kiwi, licorice, magnolia, olive, peppermint, philodendron, salvia, sasa albo-marginata, natural isoflavonoids, soy isoflavones, and natural essential oils.

In one embodiment, the composition contains metals such as metal ions, metal salts, metal complexes, fine metal powders, fine metal coated fibers and fabrics of synthetic or natural origin, or fine metal fibers. Examples of such metals include, but are not limited to, zinc, copper, aluminum, gold, silver, titanium. The metal ions provide benefits such as antimicrobial, anti-inflammatory, and/or sebum-reduction effects.

Other active agents include those commonly used as for topical treatment and in cosmetic treatment of skin tissues, such as topical antibiotics for wounds, topical antifungal drugs to treat fungal infections of the skin, and antipsoriatic drugs to treat psoriatic lesions of the skin.

Examples of antifungal drugs include but are not limited to miconazole, econazole, ketoconazole, sertaconazole, itraconazole, fluconazole, voriconazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and salts and prodrugs thereof. In one embodiment, the antifungal drugs are an azole, an allylamine, or a mixture thereof.

Examples of antibiotics (or antiseptics) include but are not limited to mupirocin, neomycin sulfate bacitracin, polymyxin B, 1-ofloxacin, tetracyclines (chlortetracycline hydrochloride, oxytetracycline-10 hydrochloride and tetrachcycline hydrochloride), clindamycin phosphate, gentamicin sulfate, metronidazole, hexylresorcinol, methylbenzethonium chloride, phenol, quaternary ammonium compounds, tea tree oil, and their cosmetically acceptable salts and prodrugs.

Examples of antimicrobials include but are not limited to salts of chlorhexidine, such as iodopropynyl butylcarbamate, diazolidinyl urea, chlorhexidene digluconate, chlorhexidene acetate, chlorhexidene isethionate, and chlorhexidene hydrochloride. Other cationic antimicrobials may also be used, such as benzalkonium chloride, benzethonium chloride, triclocarbon, polyhexamethylene biguanide, cetylpyridium chloride, methyl and benzothonium chloride. Other antimicrobials include, but are not limited to: halogenated phenolic compounds, such as 2,4,4′,-trichloro-2-hydroxy diphenyl ether (Triclosan); parachlorometa xylenol (PCMX); and short chain alcohols, such as ethanol and propanol. In one embodiment, the alcohol is preferably at a low concentration (e.g., less than about 10% by weight of the composition, such as less than 5% by weight of the composition) so that it does not cause undue drying of the skin.

Examples of antipsoriatic drugs or drugs for seborrheic dermatitis treatment include, but are not limited to, corticosteroids (e.g., betamethasone dipropionate, betamethasone valerate, clobetasol propionate, diflorasone diacetate, halobetasol propionate, triamcinonide, dexamethasone, fluocinonide, fluocinolone acetonide, halcinonide, triamcinolone acetate, hydrocortisone, hydrocortisone venerate, hydrocortisone butyrate, aclometasone dipropionte, flurandrenolide, mometasone furoate, methylprednisolone acetate), methotrexate, cyclosporine, calcipotriene, anthraline, shale oil and derivatives thereof, elubiol, ketoconazole, coal tar, salicylic acid, zinc pyrithione, selenium sulfide, hydrocortisone, sulfur, menthol, and pramoxine hydrochloride, and salts and prodrugs thereof.

Examples of anti-viral agents for viral infections such as herpes, include, but are not limited to, imiquimod and its derivatives, podofilox, podophyllin, interferon alpha, acyclovir, famcyclovir, valcyclovir, reticulos and cidofovir, and salts and prodrugs thereof.

Examples of anti-inflammatory agent, include, but are not limited to, suitable steroidal anti-inflammatory agents such as corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and salts are prodrugs thereof. A second class of anti-inflammatory agents which is useful in the compositions of the present invention includes the nonsteroidal anti-inflammatory agents.

Other active agents include, but are not limited to, wound healing enhancing agents, such as recombinant human platelet-derived growth factor (PDGF) and other growth factors, ketanserin, iloprost, prostaglandin E₁ and hyaluronic acid, scar reducing agents such as mannose-6-phosphate, analgesic agents, anesthetics, hair growth enhancing agents such as minoxadil, hair growth retarding agents such as eflornithine hydrochloride, anticancer agents, endocrine and metabolic medication, neurologic medications, vasoconstrictors, vasodilators, and biologics such as proteins, peptide, and enzymes.

Use of Composition with Device

In one embodiment, the active agent or composition is coated on (i) at least a portion of the skin-contacting surface, (ii) at least a portion of one or more of the stratum-corneum piercing microprotrusions, or (iii) at least a portion of the skin-contacting surface and at least a portion of one or more of the stratum-corneum piercing microprotrusions prior to application to the skin. In this embodiment, when device is applied onto the skin, it transfers at least a portion of the active agent or composition onto the same area of the skin that is being pierced. In one embodiment, the microprotrusion member is affixed to a patch.

In one embodiment, the device includes a reservoir containing the composition, the skin-contacting surface has at least one opening, and the reservoir is in communication with the at least one opening such that the composition can move from the reservoir, through the at least one opening, and onto the skin.

In one embodiment, the device includes a reservoir containing the composition, wherein at least one of the microprotrusions is hollow and the reservoir is in communication with the at least one hollow microprotrusion such that the composition can move from the reservoir and through the microprotrusion into the skin. In one embodiment, the composition moves through the at least one hollow microprotrusion while the at least one hollow microprotrusion is in the skin.

In one embodiment, the device is arranged to deliver from about 0.001 to about 1 ml, such as from about 0.1 to about 0.2 ml of the composition. The device may deliver only one dose of composition or multiple dosages.

In one embodiment, the active agent and/or composition is applied to the skin proximate to the time of the piercing the stratum corneum of the skin with the stratum corneum-piercing device (e.g., within about an hour before or after the piercing, such as within about fifteen minutes or within about five minutes).

In one embodiment, the composition includes an anti-coagulant, such as citric acid and salts thereof, aspirin, EDTA, dextrin, and sodium sulfate.

Product

In one embodiment, the device of the present invention and its companion products are packaged together and marketed as a kit. The examples of the items in the kit may include, but are not limited to, the device including a microprotrusion member, a predetermined number of replaceable microprotrusion members (such as the replaceable microprotrusion tips/attachments), a topical treatment composition in a suitable container/dispenser (such as a tube, a bottle, a pump, a jar, a dropper, a or unit-dose dispenser) to be used before, during, or after the stratum-corneum piercing device application. The kit may also include the energy devices (device to generate therapeutic light, electric, magnetic, electromagnetic, acoustic, thermal, mechanical energies). Additionally, the kit may also contain a cleansing product to be used to sanitize/sterilize the skin prior to the device application. The kit may also include a film forming composition or bandage to be used after treatment to protect the treated skin site and to enhance the therapeutic efficacies for the treated skin.

Methods of Use

The present invention is useful in treating a skin disorder, in particular, the surface of the skin of the face. The microprotrusion may be pushed against the surface of the skin by force such as rubbing, manual direct pressure, or through the use of an implement. In one embodiment, the implement contains at least one member (e.g., a spring or other potential energy storage element) to control the amount of force.

By piercing the stratum corneum, the skin is disrupted. By only piercing the stratum corneum and/or other layers of the epidermis, the user does not feel pain, trauma (e.g., bleeding and swelling), and/or other discomfort of the viable dermis being penetrated. Compositions, especially those with active agents, can be transported through the disrupted skin. The treatment may be localized, such that the target site of a pimple or other blemish, a wrinkle, a razor bumps/ingrown hairs, a herpes sore, a skin infection, an age-spot, or any other skin disorder.

As mentioned throughout the detailed description, there are many means for using the devices disclosed to obtain multiple benefits. For example, the microprotrusion member may be used with or without an implement, a composition containing an active agent may be placed on the treatment site prior to, during, or after treatment, the microprotrusion member may be coated with a composition containing an active agent, and the benefits derived from the invention may include treating acne, scars, wrinkles, PIH, or other skin disorders. In one embodiment, the treatment is substantially painless and does not cause scarring or bleeding. The treatment may also be used to withdraw bodily fluid such as pus from a pustule or wound exudates from the skin.

In one embodiment, the skin disorder is treated by: (a) affixing a microprotrusion to skin in need of such treatment (e.g., skin afflicted with such skin disorder); (b) applying pressure to the microprotrusion member such that one or more of the microprotrusion penetrates the stratum corneum; and (c) removing the microprotrusion member from the diseased skin. In another embodiment, the method further includes applying a composition to the skin site proximate in time to application of the microprotrusion member.

Electric Simulation

In one embodiment, the treatment is followed by a treatment with electric stimulation. Electric stimulation is known to enhance tissue repair processes such as improving wound healing and increasing collagen production. Electric stimulation is also used in needleless electric acupuncture procedures to treat diseases by application directly to body's acupuncture points on the skin. The use of an electricity-generating patch or mask to provide electric stimulation to the skin, and particularly, at the selected acupuncture points beneficial to the dermal and underlying tissues, for the purpose of treating skin diseases or disorders (such as acne, dermatitis, wrinkles, etc.) has been disclosed in U.S. Patent Application Publication No. 2004/0267169 A1 and U.S. patent application Ser. No. 11/019,557 filed Dec. 22, 2004.

In one embodiment of the present invention, prior to application of the electricity-generating patch/mask, the stratum-corneum piercing device is used to disrupt the skin at the desired location(s) of the skin, such as the selected acupuncture points, wrinkles, or acne, to reduce the electric resistance of the skin at these locations, thereby, increasing the electric current passage at the selected skin locations to enhance the desirable effect of electric stimulation.

In addition, after disrupting the stratum corneum or epidermis with the stratum-corneum disruptive device, in order to further enhance electric stimulation efficacy, the conductive carrier of the electricity-generating device may contain a relatively high concentration of cosmetically acceptable organic solvent, (e.g., glycerin, propylene glycol, or polyethylene glycol), or a non-conductive solute (e.g., low molecular weigh sugars, dextrans, or urea) to make the aqueous conductive carrier hypertonic, thus preventing the stratum corneum layer from hydrating to become more conductive. Prevention of the stratum corneum hydration reduces electric current passing through the skin except at the skin areas where the stratum corneum has been disrupted by the microprotrusion member treatment.

One example using the microprotrusion treatment for enhancing electric stimulation efficacy is to use the stratum-corneum piercing device of the present invention over a wrinkle or selected acupuncture points of the skin first, followed by application of an electricity-generating patch to cover the skin area for electric stimulation treatment. Another example is to apply the microprotrusion spot treatment device to the disease skin areas (e.g., acne, acne scar or age spots) or selected acupuncture points first, followed by application of an electricity-generating patch to cover the skin area for electric stimulation treatment. Alternatively, the microprotrusion member of the present device is built into the electricity-generating patch/mask devices, powered by a power source, such as battery, piezoelectric, electric-mechanical (e.g., a coil magnet), or by a galvanic couple, as described in U.S. patent application Ser. No. 11/019,557 filed Dec. 22, 2004, so that processes of stratum corneum disruption and electric stimulation are conducted with the same device without the need of changing devices during the treatment.

Iontophoretic Delivery of Active Agents

In one embodiment, there is one or more active agents, ionic or nonionic in nature, in the conductive carrier of the electricity-generating patch/mask that will be delivered into the skin primarily through the pathways of disrupted stratum corneum by the microprotrusion member of the present invention. One example of the active agent is Botox (Botulinum neurotoxins). Briefly, a device of the present invention applied over a wrinkle, followed by application of an electricity-generating patch to cover the skin area for electric stimulation treatment. The carrier of the electricity-generating patch contains Botox as the active agent that will be delivered into the target skin and underlying tissues by means of electrotransport (e.g., iontophoresis and electroosmosis). Alternatively, the microprotrusion member of the present device is built into the electricity-generating patch/mask devices with Botox in the carrier of the electricity-generating patch such as that described in U.S. patent application Ser. No. 11/019,557 filed Dec. 22, 2004, so that processes of stratum corneum disruption and electrotransport of Botox are conducted with the same device without the need of changing devices during the treatment.

Galvanic Microprotrusion Member

In one embodiment, the microprotrusion member or microprotrusions of the present invention are made from two dissimilar metals in contact with each other so that they form a galvanic couple, and are therefore capable of generating a galvanic current when the microprotrusion member contacts an electrolyte-containing medium. For example, the microprotrusion member may be made from a thin zinc sheet, fabricated with the manufacture methods disclosed in U.S. Pat. No. 5,983,136, 6,532,386, 6,050,988, or 6,219,574, while another metal (e.g., silver, silver-silver chloride, copper, gold) is coated on certain areas of a microprotrusion member, such as on the selected areas (e.g., the edge) of the skin-contacting surface 6, or on the microprotrusions 4 (FIG. 1).

During a skin treatment, for example, both metals of the galvanic couple (i.e., zinc and silver-silver chloride) on the microprotrusion member are in contact with an electrolyte medium (e.g., a topical composition, a body fluid such as extracellular fluid, interstitial fluid, wound exudates, sweat, and pus) and/or the skin to act as a galvanic cell (e.g., of approximately 1 volt) and to generate an electric current, going out from the zinc positive electrode, passing through the electrolyte medium and/or the skin, and returning into the silver-silver chloride negative electrode. This galvanic current may be used to provide electric stimulation and/or iontophoretic delivery of active agents into the skin via the openings/pathways across the skin barrier (i.e., stratum corneum or epidermis) created by the microprotrusions. Alternatively, the two metals forming the galvanic couple may be made to contact the third metal (e.g., titanium, or stainless steel) from which the microprotrusion member is made. For example, a zinc layer may be coated onto the selective areas of a titanium or stainless steel microprotrusion member by electric plating, electroless plating, or using a conductive ink including a zinc powder and a polymer binder. Similarly, a silver-silver chloride layer may be coated to other areas of a titanium or stainless steel microprotrusion member. The conductive metallic microprotrusion member serves as a lead to connect the galvanic elements zinc and silver-silver chloride. A galvanic current is generated when both galvanic elements coming into contact with the electrolyte medium and/or the skin during the device application.

### EXAMPLES

### Example 1

### Microprotrusion Member

Microprotrusion members containing microprotrusion arrays were produced by photochemical etching and forming using a controlled manufacturing process as described in European Patent No. 914,178 B1. The finished arrays were made of a thin sheet of titanium, and had a defined microprotrusion density of about 725 microprotrusions per cm². The microprotrusions had lengths of 145, 185 or 225 microns and had arrow-head-shaped. From this microprotrusion array sheet, a 5 mm diameter disk was cut out from such screen using a CO₂ laser.

### Example 2

### Patch

The resulting disks of microprotrusion arrays from Example 1 were affixed to an adhesive patch composed of a hydrocolloidal gel and a polyurethane film with sodium carboxymethyl cellulose adhesive (Band-Aid Advanced Healing Blister Block, Johnson & Johnson Consumer Products Company, Skillman, N.J., USA), with the microprotrusions facing away from the adhesive. The patch had a surface area of about 0.8 cm² including the 0.2 cm² microprotrusion array.

### Example 3

### Handle Implement

An implement device according to FIGS. 3-5 was made using two stainless steel compression springs (e.g., McMaster-Carr Supply Co., NJ, USA, Model, Model Gardner Spring, SS-8M for the first spring, and MC050-0330-M for second spring). The impact pressure was from about 0.5 to about 7 lbs/cm² for facial application. A slightly higher pressure was used in forearm applications.

### Example 4

### Enhancement of Active Agent Delivery

The following procedure was used to demonstrate controlled active agent delivery into skin. The microprotrusion disk of Example 1 was affixed on the desired skin site on subject's forearm or face. The implement of Example 3 was used to push the microprotrusion disk through stratum corneum with predetermined impact pressure modified by the choice of spring. The impact pressure was measured using a digital force meter (Model DFM 10, Chatillon, Greensboro, N.C.). The contact area between the implement device and skin was determined to be about 1.2 cm in diameter. The pressure per unit area was calculated from the ratio of pressure/contact area. The disk was removed immediately after the application. Both subject sensation (e.g., pain and sting) and erythema of the testing site were recorded immediately after the application, and is reported in Table 1.

TABLE 1

| | MICRO- | IMPLEMENT | SKIN |
|---|---|---|---|
| TEST | PROTRUSION | DEVICE | SENSATION |
| SUBJECT/ | LENGTH | PRESSURE | DURING |
| SKIN SITE | (MICROMETER) | (LBS/CM²) | APPLICATION |
| 1/Forearm | 225 | 8 | Slightly sting |
| 2/Forearm | 225 | 6 | Slightly sting |
| 3/Cheek (bone) | 185 | 3.2 | None |
| 4/Forearm | 185 | 4.6 | None |
| 5/Forearm | 185 | 8 | Slightly sting |
| 6/Forehead | 145 | 5 | None |

The delivery of active agents following treatment was determined by applying approximately 10 microliters of 0.10% wt/wt histamine (Sigma Aldrich, St. Louis, Mo.) on treatment test site. The reaction of the subject's skin to histamine (e.g., erythema) was recorded after 10 minutes following histamine application to the treatment site by visual inspection. Additional inspection followed if a reaction was detected at 10 minutes. Controls were run by applying histamine solution to untreated skin sites (e.g., sites not pierced by the microprotrusion members). All test sites were graded visually for the evidence of post-inflammatory hyperpigmentation (PIH) for up to at least 3 weeks. Table 2 sets forth the results of the study.

TABLE 2

| | | | ENHANCED |
|---|---|---|---|
| | | | ACTIVE |
| | | | DELIVERY |
| TEST SUBJECT/ | PRESENCE OF | PRESENCE OF | (HISTAMINE |
| SKIN SITE | ERYTHEMA | PIH | RESPONSE) |
| 1/Forearm | Present after | Yes | Yes, |
| | several hours | | consistent |
| 2/Forearm | Present after | Yes | Yes, |
| | several hours | | consistent |
| 3/Cheek | No | No | Yes, but not |
| (bone) | | | consistent |
| 4/Forearm | No | No | Yes, |
| | | | consistent |
| 5/Forearm | Present after | Yes | Yes, |
| | several hours | | consistent |
| 6/Forehead | No | No | Yes, |
| | | | consistent |

The skin's reactions to topically applied histamine following microprotrusion treatment manifested in erythema. The control test sites, however, did not result in erythema. These results, thus, indicate the microprotrusion member enhanced active agent delivery into the skin.

### Example 5

### Facial Application

Conditions and procedures in Example 4 were followed to determine the tolerance of subjects to the pressure and size of microprotrusion member when applied to facial skin. The results of the average pressure above which the users reported discomfort is reported in Table 3. Test subjects (n=10) reported substantial discomfort for an impact pressure above about 7 lbs/cm² when microprotrusion member with an area about 1 cm² was applied to human forehead skin.

TABLE 3

| | Pressure (lbs/cm2) | Pressure (lbs/cm2) |
|---|---|---|
| Location of | Applied to 1 cm2 | Applied to 2 cm2 |
| Contact | Member | Member |
| Forehead | 6.2 ± 0.8 | 3.8 ± 0.8 |
| Cheek (bone) | 4.5 ± 0.9 | 3.5 ± 1.2 |
| Cheek (soft) | 3.6 ± 0.6 | 2.3 ± 0.6 |

### Example 6

### Composition

A composition was prepared using the following components in Table 4:

TABLE 4

| CHEMICAL NAME | % WT/WT |
|---|---|
| DI water | 87.20% |
| Phenoxyethanol/parabens | 1% |
| Disodium EDTA | 0.05% |
| Dimethicone | 2% |
| Glycerin | 1.5% |
| Soy bean Seed (Soja) Extract | 5% |
| Polyacrylamide/laureth-7/isoparaffin | 3.2% |
| Butylated Hydroxytoluene (BHT) | 0.05% |

The composition was prepared as follows. The deionized water, Phenoxyethanol/parabens, and Disodium EDTA were mixed until EDTA dissolved. The Dimethicone and Glycerin were then added and mixed well until dissolved. The Soybean Seed Extract was then added and mix for ten minutes. The Polyacrylamide/laureth-7/isoparaffin and BHT were mixed together in separate beaker and then added to the aqueous batch. The mixture was then mixed for approximately one hour until a homogeneous mixture was formed. Lastly, the soymilk was homogenized into the mixture. The finished product was packaged in 1 oz tubes.

### Example 7

### Use on Acne Marks

The 185 micron length microprotrusion array disk described in the Example 1 was applied to an acne dark marks on the cheek of a subject of Fitzpatrick Skin Type VI. A dual-spring implement device described in Example 3 was applied twice onto the microprotrusion patch with an impact pressure of 4.2 lbs/cm². The disk was removed and a pea size of the composition of Example 6 was applied to the treated spot. The procedure was repeated once every other day for 21 days (on the days when the microprotrusion disk was not used, the composition was applied to the treatment site). Visible digital photos were taken at baseline and at week 3. It was found that both the dark color and size of the acne mark treated were reduced. The acne mark area had a size reduction of 34% versus baseline.

### Example 8

### Use on Wrinkles

Skin having wrinkles may also be treated. Compositions containing anti-wrinkle actives such as tretinoin (e.g., Renova from Ortho-Neutrogena, Los Angels, Calif.), retinol (e.g., Healthy Skin Anti-wrinkle Anti-blemish Cream from Neutrogena, Los Angels, Calif.), or nondenatured soy extract (e.g., Aveeno Positively Radiant Anti-wrinkle Cream from Johnson & Johnson Consumer Product Companies, Skillman, N.J.) can be post-applied daily to the microprotrusions treated skin (e.g., for at least about 4 weeks).

### Example 9

### Use to Treat Acne

A subject used the microprotrusion patch prepared from microprotrusion array or membrane described in the Example 1 and the implement of Example 3 to treat a pimple containing pus. The pimple was raised and has whitehead characteristics. An impact pressure of 5 lbs/cm² was applied by the implement device as in Example 3 to force the microprotrusions (length=185 microns) puncturing into the pimple. After releasing the microprotrusions, pus was observed to flow outward from the pimple. An anti-acne topical composition containing salicylic acid was applied to the treated pimple. Within hours, the raised pimple was visually smaller and flattened.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A method of treating acne, wherein said method comprises piercing the stratum corneum of skin in need of such treatment with a stratum corneum-piercing device, said device comprising a microprotrusion member having a skin-contacting surface and plurality of stratum corneum-piercing microprotrusions thereon, wherein said device is adapted to have an impact pressure when applied to said skin of from about 0.5 lbs/cm² to about 7 lbs/cm².

2. A method of claim 1, wherein said method further comprises applying to said skin a composition comprising an anti-acne active agent proximate to the time of said piercing the stratum corneum of said skin with said stratum corneum-piercing device.

3. A method of claim 1, wherein said device further comprises a composition comprising an anti-acne active.

4. A method of claim 3, wherein said composition is coated on (i) at least a portion of said skin-contacting surface, (ii) at least a portion of one or more of said stratum-corneum piercing microprotrusions, or (iii) at least a portion of said skin-contacting surface and at least a portion of one or more of said stratum-corneum piercing microprotrusions.

5. A method of claim 3, comprises a reservoir containing said composition, wherein said skin-contacting surface has at least one opening, and said reservoir is in communication with said at least one opening such that said composition can move from said reservoir, through said at least one opening, and onto said skin.

6. A method of claim 3, wherein said device comprises a composition reservoir containing said composition, wherein at least one of said microprotrusions is hollow, and said composition reservoir is in communication with said at least one hollow microprotrusion such that said composition can move from said composition reservoir and through said at least one hollow microprotrusion.

7. A method of claim 6, wherein said composition moves through said at least one hollow microprotrusion while said at least one hollow microprotrusion is in said skin.

8. A method of claim 1, wherein said device comprises from about 5 to about 100 microprotrusions having a length of from about 100 to about 500 microns.

9. A method of claim 1, wherein device rotates said microprotrusion member lateral to the surface of the skin surface upon contact with said skin.

10. A method of removing pus from a pimple, said method comprising piercing said pimple with a stratum corneum-piercing device, said device comprising a microprotrusion member having a skin-contacting surface and plurality of stratum corneum-piercing microprotrusions thereon, wherein said device is adapted to have an impact pressure when applied to said skin of from about 0.5 lbs/cm² to about 7 lbs/cm².

11. A method of claim 10, wherein said device comprises a collection reservoir for containing pus removed from said pimple.

12. A method of claim 11, wherein said collection reservoir comprises an absorbent material affixed to at least a portion of said skin-contacting surface.

13. A method of claim 11, wherein said skin-contacting surface has at least one openings, and said collection reservoir is in communication with said at least one opening such that said pus can move from said pimple, through said at least one opening, and into said collection reservoir.

14. A method of claim 11, wherein at least one of said microprotrusions is hollow, and said collection reservoir is in communication with said at least one hollow microprotrusion such that said pus can move from said pimple, through said at least one microprotrusion, and into said collection reservoir.

15. A method of claim 10, wherein said method further comprises applying to said skin a composition comprising an anti-acne active agent proximate to the time of said piercing said pimple with said stratum corneum-piercing device.

16. A method of claim 10, wherein said device comprises from about 5 to about 100 microprotrusions having a length of from about 100 to about 500 microns.

17. A method of claim 10, wherein device rotates said microprotrusion member lateral to the surface of the skin surface upon contact with said skin.

18. A method of claim 10, wherein said device is affixed to said skin.

19. A device comprising (i) a microprotrusion member having a skin-contacting surface and plurality of stratum corneum-piercing microprotrusions thereon and (ii) a composition comprising an anti-acne agent, wherein said device is adapted to have an impact pressure when applied to said skin of from about 0.5 lbs/cm² to about 7 lbs/cm².

20. A device of claim 19, wherein said composition is coated on (i) at least a portion of said skin-contacting surface, (ii) at least a portion of one or more of said stratum-corneum piercing microprotrusions, or (iii) at least a portion of said skin-contacting surface and at least a portion of one or more of said stratum-corneum piercing microprotrusions.

21. A device of claim 19, wherein said device comprises a reservoir containing said composition, wherein said skin-contacting surface has at least one opening, and said reservoir is in communication with said at least one opening such that said composition can move from said reservoir, through said at least one opening, and onto said skin.

22. A device of claim 19, wherein said device comprises a composition reservoir containing said composition, wherein at least one of said microprotrusions is hollow, and said composition reservoir is in communication with said at least one hollow microprotrusion such that said composition can move from said composition reservoir and through said at least one microprotrusion.

23. A device of claim 19, wherein said composition moves through said at least one microprotrusion while said at least one microprotrusion is in said skin.

24. A device of claim 19, wherein said device comprises from about 5 to about 100 microprotrusions having a length of from about 100 to about 500 microns.
